(19) United States
(12) Patent Application Publication
(10) Pub. No.: US 2013/0209553 A1
(43) Pub. Date: Aug. 15, 2013

(54) EXTENDED RELEASE PHARMACEUTICAL COMPOSITIONS OF PRAMIPEXOLE

(71) Applicants: Sunilendu Bhushan Roy, Gujarat (IN); Sushrut Krishnaji Kulkarni, Gujarat (IN); Ajay Kumar Handa, Gujarat (IN); Pranav Dhirajbhai Jogani, Gujarat (IN)

(72) Inventors: Sunilendu Bhushan Roy, Gujarat (IN); Sushrut Krishnaji Kulkarni, Gujarat (IN); Ajay Kumar Handa, Gujarat (IN); Pranav Dhirajbhai Jogani, Gujarat (IN)

(73) Assignee: CADILA HEALTHCARE LIMITED, Ahmedabad (IN)

(21) Appl. No.: 13/640,181

(22) PCT Filed: Apr. 15, 2011
(86) PCT No.: PCT/IN11/00253

§ 371 Date: Dec. 18, 2012

(30) Foreign Application Priority Data
Apr. 15, 2010 (IN) 1244/MUM/2010

(51) Int. Cl. A61K 9/20; A61K 31/428

(52) U.S. Cl. CPC A61K 9/2013; A61K 31/428
USPC 424/452; 514/367; 424/465; 424/400; 427/2.21

(57) ABSTRACT

The present invention discloses an extended release pharmaceutical composition of pramipexole or salts thereof comprising at least 40% w/w of hydrogenated castor oil, and one or more pharmaceutically acceptable excipients.

### FIELD OF THE INVENTION

The present invention relates to extended release pharmaceutical compositions of pramipexole or salt thereof. The compositions of the invention comprise hydrophobic rate-controlling polymer. The invention also relates to process of making such compositions.

### BACKGROUND OF THE INVENTION

Pramipexole is a dopamine D₂ receptor agonist useful in treatment of Parkinson's disease. Pramipexole as its dihydrochloride salt is commercially available under brand name Mirapex® tablets by Pharmacia & Upjohn. These are immediate-release tablets in 0.125 mg, 0.25 mg, 0.5 mg, 1.0 mg and 1.5 mg strengths, designed for oral administration of a single tablet three times per day to provide a daily dose of 0.375 to 4.5 mg.

Chemically, Pramipexole is (S)-2-amino-4,5,6,7-tetrahydro-6-propylamino)benzothiazole, having a structure of Formula I

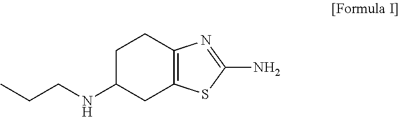

A three times daily dosing regimen for immediate-release pramipexole dihydrochloride tablets is well tolerated, but patient compliance would be much improved if a once-daily regimen were possible. In this regard, it will be noted that the primary indication for the drug, Parkinson's disease, is an affliction that becomes more prevalent with advancing age and is often accompanied by decline in memory. A once-daily regimen would be especially useful in enhancing compliance among elderly patients.

In common with other anti-Parkinson's disease drugs, pramipexole has potential to cause undesirable side effects. Side effects of pramipexole have been reported to include orthostatic hypotension, the incidence of which is dose-related. There are also reports of subjects on pramipexole medication experiencing increased somnolence, in particular “sleep attacks”. Such attacks involve a subject falling asleep while engaged in activities of daily living, including operation of a motor vehicle, sometimes resulting in accidents. Extended release compositions provide for maintains therapeutically effective plasma levels over extended period of time resulting in diminished incidences of side effects by eliminating the troughs and peaks of plasma drug concentration.

U.S. Patent Application No. 20050175691 disclose once-daily sustained release compositions of pramipexole or a pharmaceutically acceptable salts thereof having a starch, a hydrophilic polymer and a pharmaceutically acceptable excipient.

U.S. Patent Application No. 20090182024 discloses an extended release tablet formulation comprising pramipexole or a pharmaceutically acceptable salt thereof in a matrix, pregelatinized starch and an anionic polymer.

U.S. Patent Application Nos. 20090281153 and 20060198887 disclose an extended release tablet formulation comprising pramipexole or a pharmaceutically acceptable salt thereof in a matrix comprising at least one water swelling polymer other than pregelatinized starch, and wherein the formulation does not contain pregelatinized starch.

U.S. Patent Application No. 20090041844 discloses an extended release formulation of pramipexole having a reduced side effect profile when compared to immediate release formulation.

U.S. Patent Application No. 20090130197 discloses an extended release pellet comprising pramipexole or a pharmaceutically acceptable salt thereof, and at least one release-modifying excipient.

U.S. Patent Application No. 20090304794 discloses a pharmaceutical formulation of pramipexole for once-a-day administration comprising a therapeutically effective amount of pramipexole, and an osmotic agent, wherein pramipexole is released from the formulation along a pre-determined release profile.

PCT Application No. WO 2008/068778 discloses an extended release composition in the form of mini-tablets.

PCT Application No. WO 2009/117130 discloses extended release pharmaceutical formulations comprising an extended release portion and an immediate release portion, wherein the extended release portion comprising an active pharmaceutical ingredient and a wax.

There is an existing and continual need for once daily alternative extended release compositions of pramipexole or salt thereof, which can control mean plasma concentration of pramipexole in the therapeutic window and maintain steady state level of the drug below minimum toxic concentration, and thus minimize the known toxic effects of pramipexole. The inventors of the invention have surprisingly found that when pramipexole or salts thereof are formulated into extended release formulations with the use of hydrophobic rate-controlling polymer, they overcome all the encountered problems exemplified above.

### SUMMARY OF THE INVENTION

In one general aspect, there is provided an extended release pharmaceutical composition comprising pramipexole or salts thereof and one or more pharmaceutically acceptable hydrophobic rate-controlling polymers.

In another general aspect, there is provided an extended release pharmaceutical composition comprising pramipexole or salts thereof and one or more pharmaceutically acceptable rate-controlling polymers other than anionic polymers.

In another general aspect, there is provided an extended release pharmaceutical composition comprising pramipexole or salts thereof and one or more pharmaceutically acceptable hydrophobic rate-controlling polymers, wherein said composition is administered once daily.

In another general aspect, there is provided an extended release pharmaceutical composition comprising a core of pramipexole or salts thereof and one or more pharmaceutically acceptable hydrophobic rate-controlling polymers, wherein said core is functionally coated with one or more rate-controlling polymers.

In another general aspect, there is provided a matrix-type extended release pharmaceutical composition comprising pramipexole or salts thereof and one or more pharmaceutically acceptable hydrophobic rate-controlling polymers.

In another general aspect, there is provided an extended release pharmaceutical composition comprising multiple-unit particles comprising pramipexole or salts thereof and one or more pharmaceutically acceptable hydrophobic rate-controlling polymers.

Embodiments of the pharmaceutical composition may include one or more of the following features. For example, the pharmaceutical composition may further include one or more pharmaceutically acceptable excipients. The pharmaceutically acceptable excipients may include one or more binders, fillers, lubricants, disintegrants, glidants and the like.

In another general aspect, there is provided an extended release pharmaceutical composition of pramipexole or salts thereof comprising one or more pharmaceutically acceptable hydrophobic rate-controlling polymers and one or more pharmaceutically acceptable excipients, wherein said composition exhibits a dissolution profile of pramipexole or salts thereof such that at least 80% of pramipexole or salt thereof is released from about 12 hour to about 24 hour after administration.

In another general aspect, there is provided an extended release pharmaceutical composition of pramipexole or salts thereof comprising one or more pharmaceutically acceptable hydrophobic rate-controlling polymers and one or more pharmaceutically acceptable excipients, wherein said composition retains at least 80% of the potency of pramipexole or salts thereof after storage for three months at 40° C. and 75% relative humidity.

Embodiments of the pharmaceutical composition may include one or more of the following features. For example, the pharmaceutical composition may further include one or more pharmaceutically acceptable excipients. The pharmaceutically acceptable excipients may include one or more binders, fillers, lubricants, disintegrants, glidants and the like.

In another general aspect, there is provided a process for preparing an extended release pharmaceutical composition of pramipexole or salts thereof, which process comprises of mixing pramipexole or salts thereof with one or more hydrophobic rate-controlling polymers optionally with other pharmaceutically acceptable excipients and forming the mixture thus obtained into pharmaceutical dosage form.

Embodiments of the pharmaceutical composition may include one or more of the following features. For example, the pharmaceutical composition may further include one or more pharmaceutically acceptable excipients. The pharmaceutically acceptable excipients may include one or more binders, fillers, lubricants, disintegrants, glidants and the like.

In another general aspect, there is provided a method of treating Parkinson's disease in patients, wherein the method comprises administering an extended release pharmaceutical composition of pramipexole or salts thereof.

Embodiments of the pharmaceutical composition may include one or more of the following features. For example, the pharmaceutical composition may further include one or more pharmaceutically acceptable excipients. The pharmaceutically acceptable excipients may include one or more binders, fillers, lubricants, disintegrants, glidants and the like.

The details of one or more embodiments of then invention are set forth in the description below. Other features, objects and advantages of the invention will be apparent from the description.

### DETAILED DESCRIPTION OF THE INVENTION

The inventors of the present invention have surprisingly found that when pramipexole or salts thereof is formulated into extended release composition using one or more hydrophobic rate-controlling polymers, it prevents peak trough fluctuations in plasma levels and maintains therapeutically effective plasma levels of pramipexole or salts thereof over an extended period of time resulting in diminished incidences of side effects and increase in patient compliance. In particular, the inventors have found that it is possible to develop alternate formulations of pramipexole or salt thereof to persuade aforesaid objectives using relatively large amount of hydrophobic rate-controlling polymers.

Thus, the present invention provides extended release pharmaceutical compositions comprising pramipexole or salts thereof, one or more hydrophobic rate-controlling polymers and one or more pharmaceutically acceptable excipients.

In an embodiment, the extended release pharmaceutical composition of pramipexole or salts thereof in accordance with the present invention exhibits no significant difference in both rate and extent of absorption of pramipexole or salt thereof as compared to extended release formulation of pramipexole marketed under trade name Mirapex® ER.

In a further embodiment, the extended release pharmaceutical composition of pramipexole or salts thereof exhibits a dissolution profile which is suitable for once a day dosage regimen.

In a yet further embodiment the extended release composition exhibits a dissolution profile of pramipexole or salts thereof such that at least 80% of pramipexole or salt thereof is released from about 12 hour to about 24 hour when measured in phosphate buffer of pH 6.8 using USP dissolution apparatus I (Basket method) at 100 rpm.

In a still further embodiment, the extended release composition of the present invention exhibits a dissolution profile of pramipexole or salts thereof such that at least 30% pramipexole is released in 4 hours, at least 40% of pramipexole is released in 8 hours, at least 60% pramipexole is released in 12 hours, and at least 70% pramipexole is released in 24 hours when measured in phosphate buffer of pH 6.8 using USP dissolution apparatus I (Basket method) at 100 rpm.

The term “extended release” as used herein can be used synonymously with sustained release, controlled release, modified release and delayed release.

The term “pramipexole” used throughout the specification refers to not only pramipexole per se, but also its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof.

Suitable hydrophobic rate-controlling polymers include one or more of glyceride (e.g., glyceryl behenate, glyceryl trimyristate, glyceryl trilaurate, glyceryl tristearate, glyceryl monostearate, glyceryl palmitostearate, or glyceryl triacetate), stearic acid, vegetable oil and its derivatives (e.g. hydrogenated castor oil, a hydrogenated vegetable oil), a water insoluble cellulose (e.g., ethyl cellulose, cellulose acetate, cellulose acylate, cellulose diacylate, cellulose triacylate, cellulose acetate butyrate, cellulose acetate propionate, nitrocellulose, cellulose diacetate, or cellulose triacetate), a wax or a wax-like substance (e.g., carnauba wax, cetyl esters wax, beeswax, castor wax, cationic emulsifying wax, cetrimide emulsifying wax, an emulsifying wax, microcrystalline wax, a nonionic wax, a nonionic emulsifying wax, paraffin, petroleum wax, petroleum ceresin wax, spermaceti wax, white wax, or yellow wax), a fat, an oil, a fatty acid, an emulsifier, a modified starch, a fatty alcohol, a protein (e.g., zein), shellac, or a polymer (e.g., a polyolefin, a polyurethane, a polyvinylchloride, a polyvinyl acetate, an acrylic acid polymer, a methacrylic acid polymer); cetostearyl alcohol, stearyl alcohol; and the like. The hydrophobic rate-controlling polymer may be added intragranularly or extragranularly or both. The hydrophobic polymer within the matrix core acts as a release rate-controlling agent.

The compositions of the present invention may comprise rate-controlling polymers other than water swelling anionic polymers.

The present invention also contemplates pharmaceutical compositions which comprises hydrophobic rate-controlling polymers, and said composition being free of anionic and/or water soluble polymers.

The preferred amount of hydrophobic rate-controlling polymer in the extended release composition is at least 40% w/w.

Particularly preferred hydrophobic rate-controlling polymer is selected from vegetable oil or its derivatives such as hydrogenated castor oil and fatty alcohols such as stearyl alcohol or mixture thereof.

In a preferred embodiment, the ratio of amount of hydrogenated castor oil to pramipexole or salts thereof ranges from 20:1 to 200:1.

In a further preferred embodiment, the ratio of amount of stearyl alcohol to pramipexole or salts thereof ranges from 2:1 to 20:1.

The extended release pharmaceutical composition of pramipexole also remains stable over the storage period; in particular, the composition retains at least 80% of the potency of pramipexole or salts thereof in the said pharmaceutical composition after storage for three months at 40° C. and 75% relative humidity.

Suitable dosage form comprises one or more of tablets, multilayered tablets, capsules, caplets, pellets, granules, spheroids, beads, minitablets in capsule, pellets in capsule, granules or powder in capsule, powder. Further the powder or granules can be suspended to give a pharmaceutically acceptable oral suspension.

The extended release composition of pramipexole or its salt is preferably developed into dosage forms such as matrix-tablets/granules/pellets, coated tablets/granules/pellets or multiple unit particles which can be filled into capsules or compressed to form tablets.

In an embodiment, the extended release composition is not particularly limited as long as it is an oral preparation. For example, tablets, granules, fine granules, pellets, mini/micro tablets, capsules and the like can be manufactured in the present invention. Capsules can be packed with one or more tablets, granules or fine granules based on the matrix type extended release preparation according to the present invention. For example, hard capsules can be packed with multiple small-diameter mini-tablets based on the matrix type extended release preparation, or with the aforementioned granules or fine granules based on the matrix type extended release preparation, or with both tablets based on the matrix extended release preparation and granules or fine granules based on the matrix type extended release preparation. The matrix type extended release preparation can also be given a film coating as necessary.

In an embodiment, the extended release pharmaceutical composition comprises multiple-unit particles comprising pramipexole or salts thereof and one or more pharmaceutically acceptable hydrophobic rate-controlling polymers.

In a further embodiment, the extended release pharmaceutical composition comprises multiple-unit particles comprising pramipexole or salts thereof coated with one or more pharmaceutically acceptable hydrophobic rate-controlling polymers.

In a further embodiment, the extended release pharmaceutical composition comprises multiple-unit particles comprising matrix of pramipexole or salts thereof along with one or more pharmaceutically acceptable rate-controlling polymers, and optionally said multiple-unit particles are further coated with one or more hydrophobic rate-controlling polymers.

In a further embodiment, the extended release pharmaceutical composition comprises multiple-unit particles comprising inert core made up of water soluble material (e.g. lactose) or water insoluble material (e.g. microcrystalline cellulose) coated with consecutive or alternate layers of pramipexole and one or more hydrophobic rate-controlling polymer/s.

The extended release composition can also be given a film coating as necessary. It should be noted that the presence or absence of a hydrophilic film coating on the extended release preparation according to the present invention has very little effect on the dissolution profile of pramipexole or salt thereof.

The ‘non-functional coating’ or ‘film coating’ may comprise polymers like hydroxypropyl cellulose, hydroxyethyl cellulose, hydroxypropyl methylcellulose, polyvinyl pyrrolidone, copolymer of vinyl pyrrolidone and vinyl acetate; plasticizers like polyethylene glycol, triacetin, dibutyl sebecate and diethyl tartrate; opacifying agents like titanium dioxide and talc; and coloring agents. Examples of such non-functional coats are commercially available Opadry® compositions.

The pharmaceutical compositions as described herein may be prepared by processes known to the person having ordinary skill in the art of pharmaceutical technology such as direct compression, wet or dry granulation, slugging, hot melt granulation, hot melt extrusion, fluidized bed granulation, spray granulation, extrusion-spheronization, spray drying and solvent evaporation.

In an embodiment, the extended release compositions may be prepared by mixing pramipexole or salts thereof with one or more rate-controlling polymers along with one or more pharmaceutically acceptable excipients to form a blend. The blend can be lubricated and formulated into suitable dosage form.

In a further embodiment, the extended release compositions may be prepared by mixing and granulating pramipexole or salts thereof with one or more hydrophobic rate-controlling polymers along with one or more pharmaceutically acceptable excipients to form granules. The granules can be dried. The dried granules can be milled, mixed with other pharmaceutically acceptable excipients, lubricated and formulated into suitable dosage form. Further, the dosage form can be functionally coated with one or more rate-controlling polymers.

In a further embodiment, the process of manufacturing an extended release pharmaceutical composition of pramipexole or salts thereof comprises—providing a matrix and/or coated core comprising pramipexole or salts thereof, one or more hydrophobic rate-controlling polymer/s and one or more pharmaceutically acceptable excipients.

In a further embodiment, the process of preparing extended release pharmaceutical composition comprising multiple-unit particles, which process comprises steps of—

(a) coating inert core made up of water soluble (e.g. lactose or sugar) or water insoluble material (e.g. microcrystalline cellulose) with pramipexole or salts thereof with one or more rate-controlling polymer to form drug coated pellets,
(b) optionally providing one or more layer of non-functional coating or functional, coating of hydrophobic rate-controlling polymer over the drug coated pellets,
(c) filing the pellets prepared in step (b) in hard gelatin capsules.

The extended release pharmaceutical composition comprising pramipexole or salts thereof further may comprise additional anticonvulsant agent, which can be selected from one or more active agent/s selected from therapeutic category of antipsychotic agents, anticonvulsants and antidepressant agents, typical antipsychotics and atypical antipsychotics.

The pharmaceutically acceptable excipients may include one or more binders, fillers, lubricants, disintegrants, glidants and the like.

Suitable fillers may include one or more of microcrystalline cellulose, starch, dibasic calcium phosphate, tribasic calcium phosphate, calcium carbonate, dextrose, kaolin, magnesium carbonate, magnesium oxide; sugars such as lactose or sucrose; sugar alcohols such as mannitol, sorbitol, erythritol and the like.

Suitable disintegrants may include one or more of croscarmellose sodium, sodium starch glycolate, pregelatinized starch, sodium carboxymethyl cellulose, cross-linked polyvinylpyrrolidone and the like.

Suitable binders may include one or more of hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinyl pyrrolidone, pregelatinized starch, sodium alginate, gums, synthetic resins and the like.

Suitable examples of plasticizers include, but not limited to glycerin fatty acid esters; triethyl citrate; propylene glycol; polyethylene glycol and the like.

Suitable lubricants and glidants may include one or more of talc, metallic stearates such as magnesium stearate, calcium stearate, zinc stearate; colloidal silicon dioxide, finely divided silicon dioxide, stearic acid, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; and the like

In a further embodiment, the invention provides a method for treating Parkinson's disease in patients with epilepsy, wherein method comprises administering an extended release pharmaceutical composition of pramipexole or salts thereof in accordance with the present invention.

In the context of the present invention, “Bioequivalency” is determined by a 90% Confidence Interval (CI) of between 0.80 and 1.25 for both Cₘₐₓ and AUC under USFDA regulatory guidelines, or a 90% CI for AUC of between 0.80 to 1.25 and a 90% CI for Cₘₐₓ of between 0.70 to 1.43 under the European regulatory guidelines (EMEA).

The term “confidence interval, (CI)” as used herein refers to the plain meaning known to one of ordinary skill in the art. The confidence interval refers to a statistical range with a specified probability that a given parameter lies within the range.

The term “covariance, (CV)” as used herein refers to the plain meaning known to one of ordinary skill in the art. It is a statistical measure of the variance of two random variables that are observed or measured in the same mean time period. This measure is equal to the product of the deviations of corresponding values of the two variables from their respective means.

The bioequivalence studies were carried out between Mirapex® ER (reference) and compositions of the invention (test) in fasted state. The study was monitored in terms of Cₘₐₓ, AUC, Tₘₐₓ achieved with the test product and the reference product.

The compositions of the invention exhibits pharmacokinetic profile characterized by Cₘₐₓ of about 305.33 pg/ml to about 360.67 pg/ml, Tₘₐₓ of about 9.23 h to about 11.31 h, AUCₒ₋ₜ of about 5938.12 pg·h/ml to about 7132.87 pg·h/ml, AUC_{0-inf} of about 6390.55 pg·h/ml to about 7882.41 pg·h/ml.

At 90% confidence interval; area under the concentration time curve (AUCₒ₋ₜ) and/or AUC_{0-inf}) and maximum plasma concentration (Cₘₐₓ) values of composition of the invention lies between 0.70 and 1.70 as compared to that obtained by the extended release pramipexole formulation marketed under the trade name Mirapex® ER.

The results of relative bioavailability study of pramipexole extended release composition of the invention and pramipexole formulation marketed under the trade name Mirapex® ER as demonstrated in Table 8 and 9 concludes that the extended release formulation explored in the present invention provides equivalent rate and extent of absorption with reduced Cₘₐₓ compared to pramipexole formulation marketed under the trade name Mirapex® ER. In addition, the extended release formulation provides patient compliance and eliminates the fluctuations in blood plasma drug levels and toxic effects arising therefrom induced by multiple daily dosing with conventional immediate release pramipexole formulations.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

TABLE 1

| Sr. No. | Ingredients | % w/w |
|---|---|---|
| 1 | Pramipexole dihydrochloride monohydrate | 0.90 |
| 2 | Mannitol | 2.40 |
| 3 | Corn starch | 4.99 |
| 4 | Dibasic calcium phosphate dihydrate | 22.05 |
| 5 | Lactose monohydrate | 12.37 |
| 6 | Hydrogeted castor oil | 46.31 |
| 7 | Stearyl alcohol | 3.99 |
| 8 | Isopropyl alcohol | Q.S. |
| 9 | Dibasic calcium phosphate dihydrate | 2.00 |
| 10 | Lactose anhydrous | 3.99 |
| 11 | Magnesium stearate | 1.00 |
| | Total | 100 |

### Procedure:

A mixture of pramipexole dihydrochloride monohydrate, part quantity of dibasic calcium phosphate, corn starch, mannitol, lactose monohydrate and hydrogenated castor oil was granulated using binder solution of stearyl alcohol in isopropyl alcohol. The resulting granules were blended with lactose anhydrous and remaining quantity of dibasic calcium phosphate to form a blend. The blend was then lubricated with magnesium stearate and compressed into tablets using suitable tooling.

### Example 2

TABLE 2

| | | Quantity |
|---|---|---|
| Sr. No. | Ingredients | (mg/Tablet) |
| 1 | Pramipexole dihydrochloride monohydrate | 4.5 |
| 2 | Pregelatinized starch | 5-50 |
| 3 | Microcrystalline cellulose | 10-300 |
| 4 | Hydrogenated castor oil | 5-300 |
| 5 | Magnesium stearate | 1-8 |

### Procedure:

Pramipexole dihydrochloride monohydrate, pregelatinized starch, microcrystalline cellulose and hydrogenated castor oil were mixed and the blend was lubricated with magnesium stearate and compressed into tablets using suitable tooling.

### Example 3

TABLE 3

| | | Quantity |
|---|---|---|
| Sr. No. | Ingredients | (mg/Tablet) |
| 1 | Pramipexole dihydrochloride monohydrate | 4.5 |
| 2 | Lactose | 10-300 |
| 3 | Pregelatinized starch | 5-50 |
| 4 | Glyceryl behenate | 5-300 |
| 5 | Colloidal Silicone dioxide | 0.5-5 |
| 6 | Magnesium stearate | 1-8 |
| 7 | Water | Q.S. |

### Procedure:

Pramipexole dihydrochloride monohydrate, lactose, pregelatinized starch and glyceryl behenate were mixed and granulated with water. Granules were dried and mixed with colloidal silicon dioxide. The granules were lubricated with magnesium stearate and compressed into tablets using suitable tooling.

### Example 4

TABLE 4

| | | Quantity |
|---|---|---|
| Sr. No. | Ingredients | (mg/Tablet) |
| 1 | Pramipexole dihydrochloride monohydrate | 4.5 |
| 2 | Pregelatinized starch | 5-50 |
| 3 | Microcrystalline cellulose | 10-300 |
| 4 | Stearyl alcohol | 5-300 |
| 5 | Colloidal Silicone dioxide | 0.5-5 |
| 6 | Magnesium stearate | 1-8 |
| 7 | Isopropyl alcohol | Q.S. |

### Procedure:

Pramipexole dihydrochloride monohydrate, microcrystalline cellulose, pregelatinized starch and stearyl alcohol were mixed and granulated with isopropyl alcohol. Granules were dried and mixed with colloidal silicon dioxide. The granules were lubricated with magnesium stearate and compressed into tablets using suitable tooling.

### Example 5

TABLE 5

| | | Quantity |
|---|---|---|
| Sr. No. | Ingredients | (mg/Tablet) |
| 1 | Pramipexole dihydrochloride monohydrate | 4.5 |
| 2 | Pregelatinized starch | 5-50 |
| 3 | Microcrystalline cellulose | 10-300 |
| 4 | Hydrogenated castor oil | 5-300 |
| 5 | Povidone | 1-10 |
| 6 | Colloidal Silicone dioxide | 0.5-5 |
| 7 | Magnesium stearate | 1-8 |
| 8 | Water | Q.S. |

### Procedure:

Microcrystalline cellulose, pregelatinized starch and hydrogenated castor oil were mixed. Pramipexole dihydrochloride monohydrate was dissolved in water and mixed with above prepared mixture. This semi wet mass was granulated with aqueous solution of povidone. Granules were dried and mixed with colloidal silicon dioxide. The granules were lubricated with magnesium stearate and compressed into tablets using suitable tooling.

### Example 6

### Stability Study

TABLE 6

| | Related Substances | | | | |
|---|---|---|---|---|---|
| | % known impurities | | Maximum | Total | |
| Station | Impurity 1 | Impurity 2 | unknown | Impurity | Assay |
| Initial | ND | ND | 0.04 | 0.18 | 96.4 |
| 1 month | ND | ND | 0.03 | 0.16 | 96.0 |
| 2 month | ND | ND | 0.07 | 0.44 | 95.0 |
| 3 month | ND | ND | 0.05 | 0.28 | 98.7 |

ND: Not Detected

Table 6 provides stability data of the composition of the invention when stored at 40° C. and 75% relative humidity for three months and indicates that the composition remains stable over the storage period.

### Example 7

### Dissolution Study

TABLE 7

| | | Test | Test |
|---|---|---|---|
| | Reference | (% Drug release) | (% Drug release) |
| Time (hr) | (% Drug release) | (Initial) | (After 3 months) |
| 1 | 15.6 | 23.0 | 22.6 |
| 2 | 23.8 | 31.6 | 31.0 |
| 4 | 35.6 | 40.5 | 40.4 |
| 8 | 50.4 | 53.3 | 53.9 |
| 12 | 64.2 | 64.7 | 63.0 |
| 20 | 82.2 | 82.6 | 80.4 |
| 24 | 88.8 | 85 | 87.9 |

Table 7 provides dissolution data for reference and test compositions. For determination of drug release rate, USP Type I apparatus (100 rpm) was used wherein Phosphate buffer of pH 6.8 was used as medium.

### Example 8

### Bioavailability Study

In-vivo study was conducted in healthy human volunteers to assess bioavailability of pramipexole extended release tablets (Test—composition of the invention) and Mirapex® ER (Reference—Marketed pramipexole extended release tablets)

TABLE 8
Summary of PK parameters of Reference and Test compositions under
Fasting condition

| Parameters | Unit | Reference | Test |
|---|---|---|---|
| Tₘₐₓ | Hours | 9.955 | 10.318 |
| Cₘₐₓ | pg/ml | 293.245 | 329.436 |
| AUC_{0-inf} | Hr * pg/ml | 6750.065 | 7320.665 |
| AUC₀₋ₜ | Hr * pg/ml | 5867.039 | 6632.760 |

TABLE 9
Geometric LS mean ratio and 90% CI of single dose pK parameters (Reference vs. Test)

| | | Ratio | | | |
|---|---|---|---|---|---|
| Dependent | Units | [% Ref] | CI_90_Lower | CI_90_Upper | % CV |
| Ln(AUCINF_obs) | hr * pg/ml | 109.50 | 103.18 | 116.21 | 16.69 |
| Ln(AUClast) | hr * pg/ml | 113.83 | 107.51 | 120.53 | 16.05 |
| Ln(Cmax) | pg/ml | 111.98 | 106.02 | 118.28 | 15.34 |

We claim:

1. An extended release pharmaceutical composition of pramipexole or salts thereof comprising at least 40% w/w of hydrogenated castor oil, and one or more pharmaceutically acceptable excipients.

2. The extended release pharmaceutical composition as claimed in claim 1 further containing stearyl alcohol.

3. The extended release pharmaceutical composition as claimed in claim 1, wherein the ratio of amount of hydrogenated castor oil to pramipexole or salts thereof ranges from 20:1 to 200:1.

4. The extended release pharmaceutical composition as claimed in claim 2, wherein the ratio of amount of stearyl alcohol to pramipexole or salts thereof ranges from 2:1 to 20:1.

5. The extended release pharmaceutical composition as claimed in claim 1, wherein said composition is in the form of tablet, capsule, granule, powder, pellet, caplet, minitablet, capsule filled with minitablets and/or pellets, multi-layer tablet, granules for suspension, granules or powder filled in sachet.

6. The extended release pharmaceutical composition as claimed in claim 1, wherein said composition comprises matrix of pramipexole or salts thereof in hydrogenated castor oil.

7. The extended release pharmaceutical composition as claimed in claim 1, wherein said composition is in the form of multiple-unit particles.

8. The extended release pharmaceutical composition as claimed in claim 1, wherein said composition comprises core of pramipexole or salts thereof coated with hydrogenated castor oil.

9. The extended release pharmaceutical composition as claimed in claim 1, characterized in that said composition exhibits a dissolution profile of pramipexole or salts thereof such that at least 30% of pramipexole is released in 4 hours, at least 40% of pramipexole is released in 8 hours, at least 60% pramipexole is released in 12 hours, and at least 70% pramipexole is released in 24 hours when measured in phosphate buffer of pH 6.8 using USP dissolution apparatus I at 100 rpm.

10. The extended release pharmaceutical composition as claimed in claim 1, wherein said composition is suitable for once daily administration.

11. The extended release pharmaceutical composition as claimed in claim 1, wherein said composition exhibits no significant difference in both rate and extent of absorption of pramipexole or salt thereof as compared to extended release formulation of pramipexole marketed under trade name Mirapex® ER.

12. An extended release pharmaceutical composition comprising at least 40% w/w of hydrogenated castor oil, and one or more pharmaceutically acceptable excipients, wherein said composition retains at least 80% of the potency of pramipexole or salts thereof after storage for three months at 40° C. and 75% relative humidity.

13. An extended release pharmaceutical composition comprising:
(a) about 0.01-10.0% w/w of pramipexole or salts thereof;
(b) about 1.0-8.0% w/w of stearyl alcohol.
(c) about 1.0-75.0% w/w of one or more diluent/s;
(d) at least 40.0% w/w of hydrogenated castor oil;
(e) about 0.1-3% w/w of one or more lubricant/s, and

14. The extended release pharmaceutical composition as claimed in claim 13, wherein said composition further contains about 5.0-50.0% w/w of glyceryl behenate.

15. The extended release pharmaceutical composition as claimed in claim 13, wherein said diluent comprises mannitol, corn starch, pregelatinized starch, dibasic calcium phosphate, microcrystalline cellulose or mixture thereof.

16. An extended release pharmaceutical composition of pramipexole or salts thereof comprising one or more hydrophobic polymer/s, wherein said composition is devoid of water soluble polymer.

17. A process for preparing an extended release pharmaceutical composition of pramipexole or salts thereof, which process comprises:
(a) mixing, granulating or coating pramipexole or salts thereof with hydrogenated castor oil, optionally with stearyl alcohol and other pharmaceutically acceptable excipients;
(b) forming the mixture thus obtained into pharmaceutical dosage form;
wherein the amount of hydrogenated castor oil is at least 40% w/w of the composition.

18. A process of manufacturing the extended release pharmaceutical composition as claimed in claim 1, wherein said process comprises steps of:
(a) mixing pramipexole or salt thereof with hydrogenated castor oil and one or more pharmaceutically acceptable excipients
(b) granulating or compression molding of the above mixture to form suitable dosage form.

19. A process for preparing the extended release pharmaceutical composition as claimed in claim 2, wherein said process comprises steps of
(a) mixing pramipexole or salt thereof, hydrogenated castor oil and one or more pharmaceutically acceptable excipients,
(b) granulating the mixture prepared in step (a) with stearyl alcohol to form granules,
(c) converting granules prepared in step (b) in to suitable dosage form.

20. A method for treating signs and symptoms of idiopathic Parkinson's disease, wherein said method comprises administering an extended release pharmaceutical composition of pramipexole or salts thereof comprising at least 40% w/w of hydrogenated castor oil.
